(19) United States
(12) Patent Application Publication
(10) Pub. No.: US 2022/0162520 A1
(43) Pub. Date: May 26, 2022

(54) Cymbidium Goeringii Balsam and Preparation Method and Use Thereof

(71) Applicant: Guizhou Horticultural Research Institute (Guizhou Horticultural Engineering Technology Research Cent, Guiyang (CN)

(72) Inventors: Zhilin Chen, Guiyang (CN); Chaojun Zhang, Guiyang (CN); Lan Yang, Guiyang (CN); Hongjuan Xu, Guiyang (CN); Zhihui Du, Guiyang (CN)

(73) Assignee: Guizhou Horticultural Research Institute (Guizhou Horticultural Engineering Technology Research Cent, Guiyang (CN)

(21) Appl. No.: 17/165,000

(22) Filed: Feb. 2, 2021

(30) Foreign Application Priority Data
Nov. 23, 2020 (CN) 202011321467.8

(51) Int. Cl. C11B 9/02; B01J 20/22; C11B 9/00

(52) U.S. Cl. CPC C11B 9/02; B01J 20/22; C11B 9/0061; C11B 9/0076; C11B 9/0015

(57) ABSTRACT

The present disclosure belongs to the technical field of extraction of fragrance substances, and specifically relates to Cymbidium goeringii balsam and a preparation method and use thereof. The present disclosure is implemented by cultivating multiple plants of Cymbidium goeringii with unravelling bloom collectively in a relatively small enclosed space, and enriching fragrance substances of Cymbidium goeringii in the air by lipid for adsorption. The method of the present disclosure can obtain Cymbidium goeringii balsam rich in a variety of fragrance components in a relatively short time. GC-MS detection indicates as many as 50 components. The present disclosure significantly improves the prior art in which only a small amount of fragrance components in Cymbidium goeringii can be extracted or even cannot be obtained. The present disclosure can be further used in extracting essential oils or applied directly.

### TECHNICAL FIELD

The present disclosure belongs to the technical field of extraction of fragrance substances, and specifically relates to a Cymbidium goeringii (Rchb. f.) Rchb. f. (Cymbidium goeringii) balsam and a preparation method and use thereof.

### BACKGROUND

Cymbidium goeringii is a boat orchid of the genus Cymbidium which has been cultivated in China for thousands of years. It has a pleasant fragrance and is known as “king's fragrance” and “delicate fragrance”. Sometimes the fragrance can be felt and sometimes not, since the flowers have no structure to store the fragrance, and fragrance substances are released right after synthesis. Modern essential oil extraction techniques such as distillation and critical carbon dioxide extraction all aim to extract fragrance substances from flowers. But these methods are hardly suitable for Cymbidium goeringii featuring instant synthesis and instant emission of fragrance. Moreover, every inflorescence of Cymbidium goeringii generally has only 1-2 flowers, containing a small amount of fragrance substances. If the fragrance substances are extracted by a conventional method, the cost will be extremely high. Therefore, there are no orchid essential oil products currently on the market. Lipid adsorption is a traditional balsam production method which is basically replaced by modern fragrance substance extraction technology due to its cumbersome operations and high cost.

### SUMMARY

In view of this, an objective of the present disclosure is to provide Cymbidium goeringii balsam and a preparation method and use thereof. The present disclosure extracts fragrance substances from living Cymbidium goeringii by lipids to obtain rich fragrance components with simple operations and low cost.

To achieve the above objective, the present disclosure provides the following technical solutions:

The present disclosure provides a method for preparing Cymbidium goeringii balsam, which includes the following steps: cultivating multiple plants of Cymbidium goeringii with unravelling bloom collectively in an enclosed space, and adsorbing fragrance substances of Cymbidium goeringii in the enclosed space by lipid for adsorption to obtain balsam rich in fragrance substances of Cymbidium goeringii.

Preferably, varieties of the Cymbidium goeringii may include strong aromatic Cymbidium goeringii and strong aromatic Cymbidium goeringii hybrids; where varieties of the strong aromatic Cymbidium goeringii may include Cymbidium goeringii ‘Songmei’, Cymbidium goeringii ‘Zhendie’ or Cymbidium goeringii ‘Yuhudie’, and varieties of the strong aromatic Cymbidium goeringii hybrids may include Cymbidium Shouliangmei.

Preferably, the enclosed space may include an incubator.

Preferably, the cultivating multiple plants of Cymbidium goeringii collectively in an incubator may be carried out at 24-26° C. with lighting for 12 h/d at a lighting intensity of 10,000-12,000 Lx.

Preferably, the incubator may include a small glass incubator, a small artificial climate room or a thin film greenhouse.

Preferably, the absorbing fragrance substances of Cymbidium goeringii may be implemented by placing the lipid for adsorption in a washing bottle, and pumping air in the enclosed space into the washing bottle, so that the fragrance substances of Cymbidium goeringii are absorbed by the lipid for adsorption.

Preferably, the absorbing may be carried out for 12-36 h.

Preferably, the lipid for adsorption may be a mixture of lanolin, fatty alcohol polyoxyethylene ether (AEO) and purified water in a volume ratio of 1:(1-2):(1-2).

The present disclosure also provides Cymbidium goeringii balsam prepared by the above method, where the Cymbidium goeringii balsam includes the following components: 1,6,10-dodecatrien-3-ol, 3,7,11-trimethyl-, [S-(Z)]-; hexanal; 1-hexanol; furan, 2-pentyl-; 2-octenal, (E)-; phenol, 4-methyl-; trans-.alpha.-bergamotene; styrene and 3-hexen-1-ol,

The present disclosure also provides use of Cymbidium goeringii balsam prepared by the above method or the above Cymbidium goeringii balsam in preparation of Cymbidium goeringii essential oil.

The present disclosure provides a method for preparing Cymbidium goeringii balsam, including cultivating multiple plants of Cymbidium goeringii with unravelling bloom collectively in a relatively small enclosed space, and enriching fragrance substances of Cymbidium goeringii in the air by lipid for adsorption to form balsam. The method of the present disclosure can obtain Cymbidium goeringii balsam rich in a variety of fragrance components in a relatively short time. GC-MS detection indicates as many as 50 components. The present disclosure significantly improves the prior art in which only a small amount of fragrance components in Cymbidium goeringii can be extracted or even cannot be obtained. The present disclosure can be further used in extracting essential oils or applied directly.

### DETAILED DESCRIPTION

The present disclosure provides a method for preparing Cymbidium goeringii balsam, which includes the following steps: cultivating multiple plants of Cymbidium goeringii with unravelling bloom collectively in an enclosed space, and adsorbing fragrance substances of Cymbidium goeringii in the enclosed space by lipid for adsorption to obtain balsam rich in fragrance substances of Cymbidium goeringii.

In the present disclosure, varieties of the Cymbidium goeringii are not specifically restricted and may preferably include strong aromatic Cymbidium goeringii and strong aromatic Cymbidium goeringii hybrids; where varieties of the strong aromatic Cymbidium goeringii may preferably include Cymbidium goeringii ‘Songmei’, Cymbidium goeringii ‘Zhendie’ or Cymbidium goeringii ‘Yuhudie’, and varieties of the strong aromatic Cymbidium goeringii hybrids may preferably include Cymbidium Shouliangmei. The present disclosure may be implemented by cultivating multiple plants of Cymbidium goeringii with unravelling bloom collectively in an enclosed space. The enclosed space may include preferably an incubator, more preferably a small glass incubator, a small artificial climate room or a thin film greenhouse. The cultivating multiple plants of Cymbidium goeringii collectively may be carried out at 24-26° C. with lighting for 12 h/d at a lighting intensity of 10,000-12,000 Lx. In the present disclosure, type of the incubator may be preferably adjusted according to number of Cymbidium goeringii pots. In an embodiment of the present disclosure, 42 pots of Cymbidium goeringii variety Cymbidium goeringii ‘Songmei’ with about 150 flowers are placed in a 7.68 m³ incubator (6 m×0.8 m×1.6 m), and 200 ml of lipid for adsorption was used for extraction. A mixed solution has an obvious fragrance smell after 3 d.

In the present disclosure, the absorbing fragrance substances of Cymbidium goeringii may be implemented by placing the lipid for adsorption in a washing bottle, and pumping air in the enclosed space into the washing bottle, so that the fragrance substances of Cymbidium goeringii are absorbed by the lipid for adsorption. In the present disclosure, the lipid for adsorption is preferably a mixture of lanolin, AEO and purified water in a volume ratio of 1:(1-2):(1-2), and more preferably 1:(1.2-1.5):(1.2-1.5). In the present disclosure, time for adsorbing fragrance substances of Cymbidium goeringii by the lipid for adsorption may be preferably 12-36 h. In the present disclosure, blooming Cymbidium goeringii is cultivated in a relatively small enclosed space collectively to enrich fragrance substances of Cymbidium goeringii in the air. The air is introduced into the lipid for adsorption through an air pump. When the air is introduced, lipid bubbles can be formed in lipid for adsorption to increase contact area between the air and the lipid. After the air is pumped in for a long period of time, balsam is formed.

The present disclosure also provides Cymbidium goeringii balsam prepared by the above method, where the Cymbidium goeringii balsam includes the following components: 1,6,10-Dodecatrien-3-ol, 3,7,11-trimethyl-, [S-(Z)]-; hexanal; 1-hexanol; furan, 2-pentyl-; 2-octenal, (E)-; phenol, 4-methyl-; trans-.alpha.-bergamotene; styrene and 3-hexen-1-ol, (Z)-. The Cymbidium goeringii balsam of the present disclosure includes as many as 50 components as detected by GC-MS.

The present disclosure also provides use of Cymbidium goeringii balsam prepared by the above method or the above Cymbidium goeringii balsam in preparation of Cymbidium goeringii essential oil. Each component of the above lipid for adsorption of the present disclosure is an environmentally friendly non-toxic material. The balsam of the present disclosure can be directly applied for skin care or used as a raw material to prepare essential oils.

The Cymbidium goeringii balsam and the preparation method and the use thereof as provided by the present disclosure will be described in detail below with reference to embodiments, but the embodiments cannot be understood as limiting the protection scope of the present disclosure.

### Example 1

Step (1): Cymbidium goeringii selection: strong plants of artificially cultivated strong aromatic Cymbidium goeringii variety Cymbidium goeringii ‘Songmei’ were selected with unravelling bloom.

Step (2): Collective cultivation: 42 selected pots of Cymbidium goeringii (about 150 flowers) were placed in a 7.68 m³ incubator (6 m×0.8 m×1.6 m).

Step (3): Preparation of lipid for adsorption: 50 ml of lanolin, 75 ml of AEO and 75 ml of purified water were mixed.

Step (4): Lipid adsorption: 200 ml of lipid for adsorption was poured into a 1 L washing bottle. An air pump was used to pump air into the washing bottle in the incubator. After 36 h, a lanolin balsam rich in fragrance substances of Cymbidium goeringii was obtained.

The fragrance components of the lanolin balsam obtained above were detected by GC-MS. An obtained spectrum was shown in FIG. 1, and detected components were shown in Table 1.

TABLE 1
Fragrance components of Cymbidium goeringii balsam

| | | | Relative |
|---|---|---|---|
| No. | Name of component | CAS # | content, % |
| 1 | Methylene Chloride | 000075-09-2 | 1.456595 |
| 2 | Furan, 2-ethyl- | 003208-16-0 | 0.744148 |
| 3 | 3-Pentanone | 000096-22-0 | 2.196707 |
| 4 | 1-Penten-3-one | 001629-58-9 | 1.345302 |
| 5 | Hexanal | 000066-25-1 | 10.32824 |
| 6 | 2-Ethylacrolein | 000922-63-4 | 1.596361 |
| 7 | o-Xylene | 000095-47-6 | 0.8949 |
| 8 | Heptanal | 000111-71-7 | 0.816645 |
| 9 | Eucalyptol | 000470-82-6 | 0.725714 |
| 10 | 2-Hexenal, (E)- | 006728-26-3 | 0.957519 |
| 11 | Furan, 2-pentyl- | 003777-69-3 | 5.370348 |
| 12 | 1-Pentanol | 000071-41-0 | 0.332369 |
| 13 | Styrene | 000100-42-5 | 3.579807 |
| 14 | Octanal | 000124-13-0 | 0.448351 |
| 15 | 1-Octen-3-one | 004312-99-6 | 0.621094 |
| 16 | Trifluoroacetyl-lavandulol | 028673-24-7 | 0.385177 |
| 17 | 2-Penten-1-ol, (Z)- | 001576-95-0 | 0.539089 |
| 18 | 2-Heptenal, (Z)- | 057266-86-1 | 2.337259 |
| 19 | 1-Hexanol | 000111-27-3 | 7.568553 |
| 20 | 3-Hexen-1-ol, (Z)- | 000928-96-1 | 3.130382 |
| 21 | Nonanal | 000124-19-6 | 1.07352 |
| 22 | 1,3-Hexadiene, 3-ethyl-2-methyl- | 061142-36-7 | 2.481913 |
| 23 | 2-Octenal, (E)- | 002548-87-0 | 4.291833 |
| 24 | 1-Octen-3-ol | 003391-86-4 | 0.694748 |
| 25 | 1-Heptanol | 000111-70-6 | 0.664373 |
| 26 | 2,4-Heptadienal, (E,E)- | 004313-03-5 | 1.264649 |
| 27 | 1,3,4,6-Hexanetetrone, 1-(4- | 058330-10-2 | 2.62468 |
| | methylphenyl)-6-phenyl- | | |
| 28 | 2,4-Heptadienal, (E,E)- | 004313-03-5 | 1.685622 |
| 29 | Cycloheptanol | 000502-41-0 | 0.585809 |
| 30 | 3,5-Octadien-2-one, (E,E)- | 030086-02-3 | 0.318412 |
| 31 | 2-Nonenal, (E)- | 018829-56-6 | 0.296417 |
| 32 | 1-Octanol | 000111-87-5 | 0.516856 |
| 33 | Bicyclo[3.1.1]hept-2-ene, 2,6- | 017699-05-7 | 0.976564 |
| | dimethyl-6-(4-methyl-3- | | |
| | pentenyl)- | | |
| 34 | trans-.alpha.-Bergamotene | 013474-59-4 | 3.830259 |
| 35 | 2-Octen-1-ol, (E)- | 018409-17-1 | 1.21947 |
| 36 | 2-Octen-1-ol, (E)- | 018409-17-1 | 1.192945 |
| 37 | Phenethylamine, N- | 010538-85-9 | 0.215801 |
| | methyl-.beta.,3,4- | | |
| | tris(trimethylsiloxy)- | | |
| 38 | (5-Ethylcyclopent-1- | 036431-59-1 | 0.783853 |
| | enyl)methanol | | |
| 39 | 1,3-Cyclooctadiene | 001700-10-3 | 0.166615 |
| 40 | Heptadecane | 000629-78-7 | 0.40517 |
| 41 | 8-Heptadecene | 002579-04-6 | 1.76087 |
| 42 | 3,4-Dimethylbenzyl alcohol | 006966-10-5 | 0.790444 |
| 43 | Benzyl Alcohol | 000100-51-6 | 0.903693 |
| 44 | Phenol, 2-methoxy-4-methyl- | 000093-51-6 | 0.379562 |
| 45 | Acridin-9-yl-(2-methoxy-5-nitro- | 1000317-46-6 | 0.74045 |
| | phenyl)-amine | | |
| 46 | 2-Cyclohexen-1-one | 000930-68-7 | 0.675659 |
| 47 | 1,6,10-Dodecatrien-3-ol, 3,7,11- | 000142-50-7 | 18.01452 |
| | trimethyl-, [S-(Z)]- | | |
| 48 | Phenol, 4-methyl- | 000106-44-5 | 3.993174 |
| 49 | Hexadecanoic acid, methyl ester | 000112-39-0 | 0.733956 |
| 50 | Vanillin | 000121-33-5 | 1.343603 |

### Example 2

Step (1): Cymbidium goeringii selection: artificially cultivated strong aromatic Cymbidium goeringii hybrid variety Cymbidium Shouliangmei at a full-bloom stage was selected.

Step (2): Collective cultivation: three-year-old Cymbidium Shouliangmei flowering finished seedlings were collectively cultivated in a 30 m×6 m thin film greenhouse with sunshade nets. 3 seedbed racks with a width of 1.2 m and a length of 28 m were set up in the greenhouse. A total number of 840 pots were placed on the racks.

Step (3): Preparation of lipid for adsorption: 300 ml of lanolin, 360 ml of AEO and 360 ml of purified water were mixed.

Step (4): lipid adsorption: 1,020 ml of the above lipid for adsorption was poured into a 5 L washing bottle. The washing bottle was placed in the greenhouse at 6 am with one bottle at each of the sites 6 m, 12 m, 18 m and 24 m away from entrance of the greenhouse. An air pump was used to pump air into the washing bottle until 10 pm (after pumping for 16 h) to obtain a lanolin balsam with Cymbidium goeringii fragrance. In this experiment, fragrance components were not detected, but sensory evaluation was carried out. It was determined that obtained balsam had a strong Cymbidium goeringii fragrance.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A method for preparing Cymbidium goeringii balsam, comprising the following steps:
cultivating multiple plants of Cymbidium goeringii with unravelling bloom collectively in an enclosed space, and adsorbing fragrance substances of Cymbidium goeringii in the enclosed space by lipid for adsorption to obtain balsam rich in fragrance substances of Cymbidium goeringii.

2. The method according to claim 1, wherein varieties of the Cymbidium goeringii comprise strong aromatic Cymbidium goeringii and strong aromatic Cymbidium goeringii hybrids; wherein varieties of the strong aromatic Cymbidium goeringii comprise Cymbidium goeringii ‘Songmei’, Cymbidium goeringii ‘Zhendie’ or Cymbidium goeringii ‘Yuhudie’, and varieties of the strong aromatic Cymbidium goeringii hybrids comprise Cymbidium Shouliangmei.

3. The method according to claim 1, wherein the enclosed space comprises an incubator.

4. The method according to claim 3, wherein cultivating multiple plants of Cymbidium goeringii collectively in the incubator is carried out at 24-26° C. with lighting for 12 h/d at a lighting intensity of 10,000-12,000 Lx.

5. The method according to claim 3, wherein, the incubator comprises a small glass incubator, a small artificial climate room or a thin film greenhouse.

6. The method according to claim 1, wherein, the absorbing fragrance substances of Cymbidium goeringii is implemented by placing the lipid for adsorption in a washing bottle, and pumping air in the enclosed space into the washing bottle, so that the fragrance substances of Cymbidium goeringii are absorbed by the lipid for adsorption.

7. The method according to claim 6, wherein, time of the absorbing is 12-36 h.

8. The method according to claim 1 or claim 6, wherein, the lipid for adsorption is a mixture of lanolin, fatty alcohol polyoxyethylene ether (AEO) and purified water in a volume ratio of 1:(1-2):(1-2).

9. Cymbidium goeringii balsam prepared by the method according to any one of claims 1-8, wherein, the Cymbidium goeringii balsam comprises the following components: 1,6,10-dodecatrien-3-ol, 3,7,11-trimethyl-, [S-(Z)]-; hexanal; 1-hexanol;
furan, 2-pentyl-; 2-octenal, (E)-; phenol, 4-methyl-; trans-.alpha.-bergamotene; styrene and 3-hexen-1-ol, (Z)-.

10. Use of Cymbidium goeringii balsam prepared by the method according to any one of claims 1-8 or the Cymbidium goeringii balsam according to claim 9 in preparation of Cymbidium goeringii essential oil.

1. A method for preparing Cymbidium goeringii balsam, comprising the following steps: cultivating multiple plants of Cymbidium goeringii with unravelling bloom collectively in an enclosed space, and adsorbing fragrance substances of Cymbidium goeringii in the enclosed space by lipid for adsorption to obtain balsam rich in fragrance substances of Cymbidium goeringii.

2. The method according to claim 1, wherein varieties of the Cymbidium goeringii comprise strong aromatic Cymbidium goeringii and strong aromatic Cymbidium goeringii hybrids; wherein varieties of the strong aromatic Cymbidium goeringii comprise Cymbidium goeringii ‘Songmei’, Cymbidium goeringii ‘Zhendie’ or Cymbidium goeringii ‘Yuhudie’, and varieties of the strong aromatic Cymbidium goeringii hybrids comprise Cymbidium Shouliangmei.

3. The method according to claim 1, wherein the enclosed space comprises an incubator.

4. The method according to claim 3, wherein cultivating multiple plants of Cymbidium goeringii collectively in the incubator is carried out at 24-26° C. with lighting for 12 h/d at a lighting intensity of 10,000-12,000 Lx.

5. The method according to claim 3, wherein, the incubator comprises a small glass incubator, a small artificial climate room or a thin film greenhouse.

6. The method according to claim 1, wherein, the absorbing fragrance substances of Cymbidium goeringii is implemented by placing the lipid for adsorption in a washing bottle, and pumping air in the enclosed space into the washing bottle, so that the fragrance substances of Cymbidium goeringii are absorbed by the lipid for adsorption.

7. The method according to claim 6, wherein, time of the absorbing is 12-36 h.

8. The method according to claim 1, wherein, the lipid for adsorption is a mixture of lanolin, fatty alcohol polyoxyethylene ether (AEO) and purified water in a volume ratio of 1:(1-2):(1-2).

9. Cymbidium goeringii balsam prepared by the method according to claim 1, wherein, the Cymbidium goeringii balsam comprises the following components: 1,6,10-dodecatrien-3-ol; hexanal; 1-hexanol; furan, 2-pentyl-; 2-octenal, (E)-; phenol, 4-methyl-; trans-.alpha.-bergamotene; styrene and 3-hexen-1-ol, (Z)-.

10. Use of Cymbidium goeringii balsam prepared by the method according to claim 1 in preparation of Cymbidium goeringii essential oil.

11. The method according to claim 6, wherein, the lipid for adsorption is a mixture of lanolin fatty alcohol polyoxyethylene ether (AEO) and purified water in a volume ratio of 1:(1-2):(1-2).

12. Cymbidium goeringii balsam prepared by the method according to claim 2, wherein, the Cymbidium goeringii balsam prepared by the method according to claim 2, wherein, the Cymbidium goeringii balsam comprises the following components: 1,6,10-dodecatrien-3-ol, hexanal; 1-hexanol; furan, 2-pentyl-; 2-octenal, (E)-; phenol, 4-methyl-; trans-alpha-bergamotene; styrene and 3-hexen-1-ol, (Z)-,

13. Cymbidium goeringii balsam prepared by the method according to claim 3, wherein, the Cymbidium goeringii balsam comprises the following components: 1,6,10-dodecatrien-3-ol; hexanal; 1-hexanol; furan, 2-pentyl-; 2-octenal, (E)-; phenol, 4-methyl-; trans-.alpha.-bergamolene; styrene and 3-hexen-1-ol, (Z)-.

17. Cymbidium goeringii balsam prepared by the method according to claim 7, wherein, the Cymbidium goeringii balsam comprises the following components: 1,6,10-dodecatrien-3-ol; hexanal; 1-hexanol; furan, 2-pentyl-; 2-octenal, (E)-; phenol, 4-methyl-; trans-.alpha.-bergamotene; styrene and 3-hexen-1-ol, (Z)-.

18. Cymbidium goeringii balsam prepared by the method according to claim 8, wherein the Cymbidium goeringii balsam comprises the following components: 1,6,10-dodecatrien-3-ol, hexanal; 1-hexanol; furan, 2-pentyl-; 2-octernal, (E)-; phenol, 4-methyl-; trans-alpha-bergamotene; styrene and 3-hexen-1-ol, (Z)-.

19. Cymbidium goeringii balsam prepared by the method according to claim 11, wherein, the Cymbidium goeringii balsam comprises the following components: 1,6,10-dodecatrien-3-ol; hexanal; 1-hexanol, furan, 2-pentyl-; 2-octenal, (E)-; phenol, 4-methyl-, trans-.alpha.-bergamotene, styrene and 3-hexen-1-ol, (Z)-.

20. Use of the Cymbidium goeringii balsam according to claim 9 in preparation of Cymbidium goeringii essential oil.
